(19) United States
(12) Patent Application Publication
(10) Pub. No.: US 2011/0282421 A1
(43) Pub. Date: Nov. 17, 2011

(54) DIAMOND NEURAL DEVICES AND ASSOCIATED METHODS

(76) Inventor: Chien-Min Sung, Tansui (TW)

(21) Appl. No.: 13/082,085

(22) Filed: Apr. 7, 2011

Related U.S. Application Data
(60) Provisional application No. 61/333,629, filed on May 11, 2010.

(51) Int. Cl. A61N 1/05; B82Y 5/00; B82Y 40/00; H01L 21/04

(52) U.S. Cl. 607/118; 438/105; 977/904; 977/891; 257/E21.049

(57) ABSTRACT

The present disclosure provides devices for neuronal growth and associate methods. In one aspect, for example, a neuronal growth device is provided including a layer of nanodiamond particles having an exposed neuronal growth surface, a doped diamond layer contacting the layer of nanodiamond particles opposite the neuronal growth surface, and a semiconductor layer coupled to the doped diamond layer opposite the layer of nanodiamond particles. In one aspect, the nanodiamond particles are substantially immobilized by the doped diamond layer.

### PRIORITY DATA

This application claims the benefit of U.S. Provisional Patent Application Ser. No. 61/333,629, filed on May 11, 2010, which is incorporated herein by reference in its entirety.

### BACKGROUND OF THE INVENTION

Growth surfaces for neurons and other cellular material has proven challenging. In adhesion of neurons, for example, too many substrates are problematic, requiring complex protein coatings to be applied to the substrates. Such protein coatings are difficult to apply and may be less desirable for use in chronic neural implants. These protein materials may be toxic at some level, and in some cases may alter the normal growth and function of surrounding neurons.

Additionally, various groups have attempted to interface neuronal material with electronic devices with little success. Materials that can provide adequate anchoring of neurons are generally not conducive to integration with electronic devices. Similarly, materials that incorporate well into electronic devices may not provide anchorage for neuronal materials without complex protein coatings.

### SUMMARY OF THE INVENTION

The present disclosure provides devices for neuronal growth and associate methods. In one aspect, for example, a neuronal growth device is provided including a layer of nanodiamond particles having an exposed neuronal growth surface, a doped diamond layer contacting the layer of nanodiamond particles opposite the neuronal growth surface, and a semiconductor layer coupled to the doped diamond layer opposite the layer of nanodiamond particles. In one aspect, the nanodiamond particles are substantially immobilized by the doped diamond layer.

The diamond layers of the present disclosure can be doped with a variety of dopants depending on the desired nature of the resulting doped diamond layer. As such, any dopant capable of incorporation into a diamond material should be considered to be within the present scope. In one aspect, however, non-limiting examples of such materials include boron-doped diamond, nitrogen-doped diamond, and the like, including combinations thereof. In one specific aspect, the doped diamond layer is a boron-doped diamond layer.

The semiconductor materials of the semiconductor layer can vary depending on the intended nature of the resulting device. As such, any known semiconductor material is considered to be within the present scope. Non-limiting examples of semiconductor layer materials can include GaN, AlN, (B, Al)N, (Ga, In)N, TlN, Al₂O₃, and the like, including combinations thereof. In another aspect, the semiconductor layer can include GaN, (Ga, In)N, and the like. Additionally, the semiconductor layer can include mixed crystal semiconductor materials. In one specific aspect, for example, the semiconductor layer can have a (B, Al, Ga, In, Tl) mixed crystal lattice. In another specific aspect, for example, the semiconductor layer can have a (B, Al, Ga, In) mixed crystal lattice.

Additionally, various techniques for coupling the semiconductor layer to the diamond layer are contemplated, and any such method should be considered to be within the present scope. In one aspect, for example, the semiconductor layer can be coupled to the diamond layer with a member selected from the group consisting of braze, solder, organic adhesives and ceramic binders and combinations thereof. In another aspect, the semiconductor layer can be formed on the diamond layer. Furthermore, in some aspects a metal layer can be disposed between the diamond layer and the semiconductor layer.

The present disclosure additionally provides methods of making a neuronal growth substrate. In one aspect such a method can include forming a layer of nanodiamond particles on a support substrate, forming a layer of diamond on the layer of nanodiamond particles opposite to the support substrate, and doping the layer of diamond. The method can further include coupling a semiconductor layer to the doped diamond layer opposite to the layer of nanodiamond particles and removing the support substrate from the layer of nanodiamond particles to expose a neuronal growth surface.

Various techniques are contemplated for forming nanodiamond particle layers. In one aspect, for example, forming the layer of nanodiamond particles on the support substrate can include contacting the support substrate to a solution containing suspended nanodiamond particles and ultrasonically agitating the solution to embed the nanodiamond particles on the support substrate. In another aspect, forming the layer of nanodiamond particles on the support substrate further includes vapor depositing the layer of nanodiamond particles on the support substrate. In yet another aspect, forming the layer of nanodiamond particles on the support substrate further includes sintering polycrystalline diamond into a polycrystalline diamond layer on the support substrate.

The semiconductor layer can be coupled to the diamond layer using a variety of techniques. In one aspect, for example, the semiconductor layer can be brazed to the diamond layer. In another aspect, the semiconductor layer can be soldered to the diamond layer. In a further aspect, the semiconductor layer can be adhered to the diamond layer with a resin or ceramic binder. In yet another aspect, the semiconductor layer can be formed on the diamond layer.

In yet another aspect of the present disclosure, a neuronal training device is provided. Such a device can include a pair of opposing electrode layers spaced apart to create a neuronal growth space there between, where each opposing electrode layer has an electrode array oriented toward the neuronal growth space such that current applied to the electrode array flows across the neuronal growth space. The device can also include spacer elements disposed between the opposing electrode layers to maintain the neuronal growth space. In another aspect, the device can include a seal member enclosing at least a portion of the neuronal growth space and operable to maintain a liquid in the neuronal growth space.

In a further aspect of the present disclosure, a neuronal growth device can include a polycrystalline diamond layer having an exposed neuronal growth surface and a semiconductor layer coupled to the polycrystalline diamond layer opposite the neuronal growth surface. In one aspect the polycrystalline diamond layer is a doped polycrystalline diamond layer.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

FIG. 1 is a cross-section view of a neuronal growth device in accordance with one embodiment of the present invention.

FIGS. 2a-d are cross-section views showing manufacturing steps of a neuronal growth device in accordance with one embodiment of the present invention.

FIG. 3 is a cross-section view of a neuronal training device in accordance with one embodiment of the present invention.

### DETAILED DESCRIPTION OF THE INVENTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a dopant” includes reference to one or more of such dopants, and reference to “the diamond layer” includes reference to one or more of such layers.

As used herein, “vapor deposited” refers to materials which are formed using vapor deposition techniques. “Vapor deposition” refers to a process of forming or depositing materials on a substrate through the vapor phase. Vapor deposition processes can include any process such as, but not limited to, chemical vapor deposition (CVD) and physical vapor deposition (PVD). A wide variety of variations of each vapor deposition method can be performed by those skilled in the art. Examples of vapor deposition methods include hot filament CVD, rf-CVD, laser CVD (LCVD), laser ablation, conformal diamond coating processes, metal-organic CVD (MOCVD), sputtering, thermal evaporation PVD, ionized metal PVD (IMPVD), electron beam PVD (EBPVD), reactive PVD, and the like.

As used herein, “chemical vapor deposition,” or “CVD” refers to any method of chemically forming or depositing diamond particles in a vapor form upon a surface. Various CVD techniques are well known in the art.

As used herein, “physical vapor deposition,” or “PVD” refers to any method of physically forming or depositing diamond particles in a vapor form upon a surface. Various PVD techniques are well known in the art.

As used herein, “diamond” refers to a crystalline structure of carbon atoms bonded to other carbon atoms in a lattice of tetrahedral coordination known as sp³ bonding. Specifically, each carbon atom is surrounded by and bonded to four other carbon atoms, each located on the tip of a regular tetrahedron. Further, the bond length between any two carbon atoms is 1.54 angstroms at ambient temperature conditions, and the angle between any two bonds is 109 degrees, 28 minutes, and 16 seconds although experimental results may vary slightly. The structure and nature of diamond, including its physical and electrical properties are well known in the art.

As used herein, “distorted tetrahedral coordination” refers to a tetrahedral bonding configuration of carbon atoms that is irregular, or has deviated from the normal tetrahedron configuration of diamond as described above. Such distortion generally results in lengthening of some bonds and shortening of others, as well as the variation of the bond angles between the bonds. Additionally, the distortion of the tetrahedron alters the characteristics and properties of the carbon to effectively lie between the characteristics of carbon bonded in sp³ configuration (i.e. diamond) and carbon bonded in sp² configuration (i.e. graphite). One example of material having carbon atoms bonded in distorted tetrahedral bonding is amorphous diamond.

As used herein, “diamond-like carbon” refers to a carbonaceous material having carbon atoms as the majority element, with a substantial amount of such carbon atoms bonded in distorted tetrahedral coordination. Diamond-like carbon (DLC) can be formed by PVD and CVD processes. Notably, a variety of other elements can be included in the DLC material as either impurities, or as dopants, including without limitation, hydrogen, sulfur, phosphorous, boron, nitrogen, silicon, tungsten, etc.

As used herein, “amorphous diamond” refers to a type of diamond-like carbon having carbon atoms as the majority element, with a substantial amount of such carbon atoms bonded in distorted tetrahedral coordination. In one aspect, the amount of carbon in the amorphous diamond can be at least about 90%, with at least about 20% of such carbon being bonded in distorted tetrahedral coordination. Amorphous diamond also has a higher atomic density than that of diamond (176 atoms/cm³). Further, amorphous diamond and diamond materials contract upon melting.

As used herein, “substrate” refers to a support surface to which various materials can be joined. The substrate can be any shape, thickness, or material, required in order to achieve a specific result, and includes but is not limited to metals, alloys, ceramics, and mixtures thereof. Further, in some aspects, the substrate may be an existing semiconductor device or wafer.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### THE INVENTION

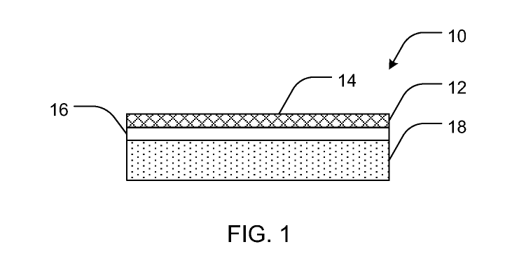

The present invention provides various neuronal growth devices, neural training devices, and associated methods. In one aspect, as is shown in FIG. 1, a neuronal growth device 10 can include a layer of nanodiamond particles 12 having an exposed neuronal growth surface 14, a doped diamond layer 16 contacting the layer of nanodiamond particles 12 opposite the neuronal growth surface 14, and a semiconductor layer 18 coupled to the doped diamond layer 16 opposite the layer of nanodiamond particles 12. Thus in some aspects such a device can provide functional integration between a growth surface and an electronic interconnect. In other aspects, the device can be used as a sensory detector.

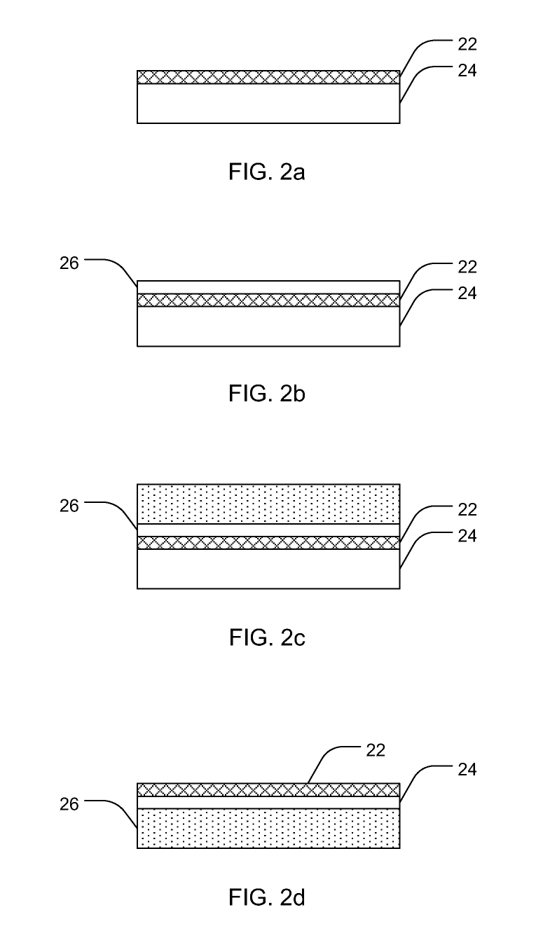

In another aspect of the present invention, as is shown in FIGS. 2a-d, a method of making a neuronal growth substrate is provided. Such a method can include forming a layer of nanodiamond particles 22 on a support substrate 24, forming a layer of diamond 26 on the layer of nanodiamond particles 22 opposite to the support substrate 24, and doping the layer of diamond 26. The method can also include coupling a semiconductor layer 28 to the doped diamond layer 26 opposite to the layer of nanodiamond particles 22 and removing the support substrate 24 from the layer of nanodiamond particles 22 to expose a neuronal growth surface 30.

Layers of nanodiamond make useful substrates for the anchorage of neurons, and as such, can promote the formation of functional neuronal networks. These nanodiamond materials can promote the attachment and growth of neurons without the requirement of using protein coatings, as is the case with other growth surfaces such as metals or ceramics. Protein coatings can increase the complexity of the substrate preparation and growth procedures. Such coatings can also be undesirable for in vivo use. Nanodiamond materials, on the other hand, are stable and non-toxic to biological organisms, thus providing a useful substrate for chronic neural implants.

Various types of nanodiamond materials are known, and any such material that can be formed into a layer and that can function as a neuronal growth surface is considered to be within the scope of the present disclosure. Nanodiamond particles can be formed using a number of techniques such as shock wave synthesis, CVD formation, and the like. In shock wave synthesis, for example, nanodiamond is formed by the detonation of TNT/RDX in a controlled environment. Nanodiamond can also be synthesized from a suspension of graphite in organic liquid at atmospheric pressure and room temperature using ultrasonic cavitation. Irradiation of graphite by high-energy laser pulses can also be used to produce nanodiamond. Other nanoparticles are also contemplated that can be used to form an anchorage surface for neuronal growth. One example of such a material is nanosized particles of cubic boron nitride (cBN).

Nanodiamond particles can then be attached to a substrate for further processing. In one aspect, for example, nanodiamond particles can be attached to the substrate by sonication. In one such process, the support substrate can be contacted to a solution containing suspended nanodiamond particles. In one aspect, the nanodiamond particles can be suspended in a bath, such as, for example, ethanol, and the substrate can be placed in the bath. The bath can then be ultrasonically agitated to embed the nanodiamond particles from the solution and onto the support substrate. The nanodiamond particles are embedded on the surface of a submerged substrate by the ultrasonic agitation. The density of nanodiamond particles embedded on the substrate is proportional to the time of agitation. Such densities can be as high as 100 billion per centimeter.

In other aspects of the present invention, a layer of nanodiamond particles can be formed directly on a substrate material. Various methods for creating a nanodiamond layer on a substrate may include, without limitation, PVD (e.g. sputtering), CVD (e.g. adamantane decomposition), and the like. Additionally, numerous methods of transferring a crystalline material from a crystalline target to a substrate can be utilized to form a nanocrystalline layer. Examples of such techniques include, without limitation, sputtering, ion beam sputtering, arc deposition with cosputtering, pulsed laser ablation, magnetron sputtering, and the like. As one specific example, transferring diamond material to a substrate can be accomplished by laser ablation of a diamond target to form nanocyrstalline particles on the substrate.

The thickness of nanodiamond layers can vary depending on the intended use of the nanodiamond and the technique whereby the layer was formed. In one aspect, however, the nanodiamond layer can be less than about 1 nm thick. In another aspect, the nanodiamond layer can be from about 1 nm thick to about 10 nm thick. In yet another aspect, the nanodiamond layer can be from about 1 nm thick to about 1 micron thick. In yet another aspect, the nanodiamond layer can be from about 10 nm thick to about 100 nm thick. In a further aspect, the nanodiamond layer can be greater than about 5 nm thick. In yet a further aspect, the nanodiamond layer can be less than about 500 nm thick.

Various support substrates can be utilized for temporarily retaining the nanodiamond layer depending on the desired construction process and conditions of the nanodiamond formation. For example, PVD and CVD deposition methods are often performed at high deposition temperatures (e.g. 900° C.). Such high temperatures can cause detrimental effects to many substrate materials, such as warping, delamination, back-conversion (e.g. Ni causes diamond to back-convert to graphite), among others. Substrates used for such processes can be chosen to minimize such detrimental effects. Sputtering and sonication procedures, however, can be accomplished at lower temperatures, thus allowing the use of a greater variety of available substrates. It should be understood, however, that any support substrate can be utilized that is capable of receiving a nanodiamond layer. One useful example of such a support substrate is a silicon material, such as a silicon wafer. Other general examples can include metals, ceramics, polymers, semiconductors, and the like.

In other aspects of the present invention, a layer of nanodiamond particles can be formed from sintered polycrystalline diamond (PCD). A sintered PCD layer can thus also be used as the growth surface for neurons. In one aspect, PCD is sintered with the aid of molten cobalt. Such a layer of PCD is thus electrically conducting, via the residue Co network within the PCD structure. One side of the PCD layer can be leached with an acid (e.g. nitric acid) to expose the nanodiamond particles for a neuronal growth surface. In yet another aspect, a layer of carbon nanotubes (CNT) can be used instead of, or in addition to, the nanodiamond particle layer. It should be noted that PCD layers formed from techniques other than sintering are also contemplated, provided the polycrystalline structure of the layer is sufficient to promote neuronal growth.

Following formation of the nanodiamond layer, a diamond layer is formed thereon. The diamond functions, among other things, as a layer that immobilizes and adds support to the nanodiamond layer. Nanodiamond particles provide effective seeding for the growth of the diamond layer. The diamond layer can be doped or undoped, depending on the intended nature of the device, as is discussed more fully herein. Various types of diamond layers are contemplated that would be useful according to aspects of the present invention. Non-limiting examples include crystalline diamond, diamond-like carbon, amorphous diamond, and the like.

It should be understood that the following is a very general discussion of diamond deposition techniques that may or may not apply to a particular aspect or application, and that such techniques may vary widely and still be within the present scope. Generally, diamond layers may be formed by any means known, including various vapor deposition techniques. Any number of known vapor deposition techniques may be used to form these diamond layers. The most common vapor deposition techniques include chemical vapor deposition (CVD) and physical vapor deposition (PVD), although any similar method can be used if similar properties and results are obtained. In one aspect, CVD techniques such as hot filament, microwave plasma, oxyacetylene flame, rf-CVD, laser CVD (LCVD), metal-organic CVD (MOCVD), laser ablation, conformal diamond coating processes, and direct current arc techniques may be utilized. Typical CVD techniques use gas reactants to deposit the diamond or DLC material in a layer, or film. These gases generally include a small amount (i.e. less than about 5%) of a carbonaceous material, such as methane, diluted in hydrogen. A variety of specific CVD processes, including equipment and conditions, as well as those used for semiconductor layers, are well known to those skilled in the art. In another aspect, PVD techniques such as sputtering, cathodic arc, and thermal evaporation may be utilized. Additionally, molecular beam epitaxy (MBE), atomic layer deposition (ALD), and the like can additionally be used. Further, specific deposition conditions may be used in order to adjust the exact type of material to be deposited, whether DLC, amorphous diamond, or crystalline diamond.

As an example, in one aspect a seeded substrate can be placed in a CVD reactor (e.g. hot filament or microwave) that is purged with methane (e.g. 1%) and trimethylboride (e.g. 1%) in hydrogen at about 40 Torr pressure. The reactor can form a plasma that decomposes methane to carbon atoms. Carbon atoms that contact nanodiamond will grow homoepitaxially. The diamond grains grow together to form a diamond layer. A dopant can be added to the reactor to dope the growing diamond layer as it forms. In one specific aspect, a boron-doped diamond layer is formed. In another aspect, a nitrogen-doped diamond layer is formed.

Various methods may be employed to increase the quality of the diamond layer that is created by vapor deposition techniques. For example, diamond quality can be increased by reducing the methane flow rate, and increasing the total gas pressure during the early phase of diamond deposition. Such measures decrease the decomposition rate of carbon and increase the concentration of hydrogen atoms. Thus a significantly higher percentage of the carbon will be deposited in a sp³ bonding configuration, and the quality of the diamond nuclei formed is increased.

In one aspect of the present invention, the diamond layer may be formed as a conformal diamond layer. Conformal diamond coating processes can provide a number of advantages over conventional diamond film processes. Conformal diamond coating can be performed on a wide variety of substrates, including non-planar substrates. A growth surface can be pretreated under diamond growth conditions in the absence of a bias to form a carbon film. The diamond growth conditions can be conditions that are conventional CVD deposition conditions for diamond without an applied bias. As a result, a thin carbon film can be formed which is typically less than about 100 angstroms. The pretreatment step can be performed at almost any growth temperature such as from about 200° C. to about 900° C., although lower temperatures below about 500° C. may be preferred. Without being bound to any particular theory, the thin carbon film appears to form within a short time, e.g., less than one hour, and is a hydrogen terminated amorphous carbon.

Following formation of the thin carbon film, the growth surface may then be subjected to diamond growth conditions to form a conformal diamond layer. The diamond growth conditions may be those conditions which are commonly used in traditional CVD diamond growth. However, unlike conventional diamond film growth, the diamond film produced using the above pretreatment steps results in a conformal diamond film that typically begins growth substantially over the entire growth surface with substantially no incubation time. In addition, a continuous film, e.g. substantially no grain boundaries, can develop within about 80 nm of growth. Diamond layers having substantially no grain boundaries may move heat more efficiently than those layers having grain boundaries.

The diamond layers according to aspects of the present invention may be of a variety of thicknesses. In one aspect, a diamond layer may be from about 10 to about 50 microns thick. In another aspect, a diamond layer may be less than or equal to about 10 microns thick. In yet another aspect, a diamond layer may be from about 50 microns to about 100 microns thick. In a further aspect, a diamond layer may be greater than about 50 microns thick. In one specific aspect, the diamond layer can be about 20 microns thick.

Additionally, in some aspects the diamond layer can be doped to form a conductive layer. Various techniques may be employed to render a diamond layer conductive. Such techniques are known to those of ordinary skill in the art. For example, various impurities can be doped into the diamond layer. Such impurities or dopants can include elements such as Si, B, P, N, Li, Al, Ga, and the like. In one specific aspect, for example, the diamond layer can be doped with boron. In another aspect, the diamond layer can be doped with nitrogen. Impurities may also include metallic particles within the diamond layer, provided they do not interfere with the function of the device.

As has been described, the nanodiamond layer allows anchorage of neurons without the need for protein coatings. In one aspect, however, a neuronal growth device can be fabricated that utilizes the diamond layer as a neuronal growth surface. By etching or otherwise processing the diamond layer to have a roughened surface, neurons are able to anchor thereto. The surface can be etched with regular structures or protrusions such as pyramids or peaks, or the surface can be etched with a more irregular structure design. It should be noted that the present scope should not be limited by the type of roughening that occurs to the diamond layer.

Following formation of the diamond layer, a semiconductor layer can be associated therewith. The semiconductor layer can be formed on the diamond layer, or alternatively the semiconductor layer can be formed separately from the diamond layer and coupled thereto. For example, in one aspect the semiconductor layer can be soldered to the diamond layer. For purposes of the present disclosure, soldering is any process utilizing a metal or a metal alloy to bind two materials together, where the metal or metal alloy has a melting temperature of from about 90° C. to about 450° C. In another aspect, the semiconductor layer can be brazed to the diamond layer. For purposes of the present disclosure, brazing is any process utilizing a metal or a metal alloy to bind two materials together, where the metal or metal alloy has a melting temperature of greater than about 450° C. In one specific aspect, the diamond layer can be coated with titanium followed by gold. A semiconductor layer can subsequently be soldered to the gold layer. In one aspect, the diamond layer can be coated with titanium and gold, and then soldered to a gallium nitride layer on a sapphire substrate. The sapphire substrate can then be laser irradiated to vaporize Ga atoms, and the sapphire substrate can be removed, leaving a gallium nitride semiconductor layer exposed.

In addition to coupling, the semiconductor layer can be formed directly on the diamond layer. Various known methods of vapor deposition can be utilized to deposit such layers. In some cases, techniques can be utilized to allow deposition to occur in a graded manner. For example, a diamond lattice (single crystal or polycrystalline) can grade to SiC or AN using various techniques. One such technique is Atomic Layer Deposition (ALD). Unlike CVD methods (e.g. MOCVD for growing GaN on SiC or sapphire) that require the reaction of gasses before depositing the products on a substrate, ALD reacts the deposition gas with the substrate and purges unreacted gas, thus leaving one layer of bonded atoms on the substrate. Using this method the diamond lattice can grade to SiC via layers of gradually decreasing carbon content. Such gradation can be based on the ratio of Si/C atoms, or it can be based on layers of Si versus layers C. In either case, ALD can control the entry of reactants as a mixture or as one composition and alternate with another composition.

Thus, in one aspect diamond can be graded to SiC, the SiC can be graded to AN, and the AN can be graded to GaN. In another aspect, diamond can grade to BN, then to AN, then to GaN, and then to InN. In all these grading situations, the manipulation of the composition is gradual so the lattice continuity is maintained, i.e. the lattice mismatch and distortion stress is spread across many layers.

Various semiconductor materials are contemplated that can be used in the devices according to aspects of the present invention. The semiconductor layer may include any material that is suitable for forming semiconductor devices. Many semiconductors materials are based on silicon, gallium, indium, and germanium. However, suitable materials for the semiconductor layer can include, without limitation, silicon, silicon carbide, silicon germanium, gallium arsenide, gallium nitride, germanium, zinc sulfide, gallium phosphide, gallium antimonide, gallium indium arsenide phosphide, aluminum phosphide, aluminum arsenide, aluminum gallium arsenide, gallium nitride, boron nitride, aluminum nitride, indium arsenide, indium phosphide, indium antimonide, indium nitride, sapphire, and composites thereof. In one aspect, however, the semiconductor layer can include silicon, silicon carbide, gallium arsenide, gallium nitride, gallium phosphide, aluminum nitride, indium nitride, indium gallium nitride, aluminum gallium nitride, or composites of these materials.

In some additional aspects, non-silicon based devices can be formed such as those based on gallium arsenide, gallium nitride, germanium, boron nitride, aluminum nitride, indium-based materials, and composites thereof. In another embodiment, the semiconductor layer can comprise gallium nitride, indium gallium nitride, indium nitride, and combinations thereof. In one specific aspect, the semiconductor material is gallium nitride. In another specific aspect, the semiconductor material is aluminum nitride.

Additionally, semiconductor materials may be of any structural configuration known, for example, without limitation, cubic (zincblende or sphalerite), wurtzitic, rhombohedral, graphitic, turbostratic, pyrolytic, hexagonal, amorphous, or combinations thereof. As has been described, the semiconductor layer may be formed by any method known to one of ordinary skill in the art. Various known methods of vapor deposition can be utilized to deposit such layers and that allow deposition to occur in a graded manner. Additionally, surface processing may be performed between any of the deposition steps described in order to provide a smooth surface for subsequent deposition. Such processing may be accomplished by any means known, such as by chemical etching, polishing, buffing, grinding, etc.

Depending on the method used to manufacture the neuronal growth device, the support substrate can be removed to expose the neuronal growth surface of the nanodiamond layer. The method of removing the support substrate can vary depending on the physical natures of the substrate material and the nanodiamond layer, as well as process preferences. For example, a silicon support substrate can be removed by etching in a concentrated NaOH solution to expose the nanodiamond layer. Removal methods can also include grinding, laser cutting, sawing, and the like. In some cases a combination of methods can be beneficial. For example, a silicon substrate can be cut or ground to remove a majority of the substrate material, followed by chemical etching to preserve the structure of the nanodiamond layer.

Semiconductor materials having different bandgaps can be deposited to form a multi-junction that can absorb different wavelengths (energy) of light. In the case of (Ga, In)N, the composition may be graded as to form a superlattice. This lattice will absorb wave lengths from red (InN) to purple (GaN). If TIN is incorporated, the lattice can detect infrared waves. On the other hand, if AlN is incorporated, ultraviolet waves can be detected. Since B, Al, Ga, In, and Tl are all Group III elements, they have similar charges and chemical properties. Their differences lies in atomic sizes, and hence bonding strength with N atoms, which is the reason for the different band gap. The B atoms of BN are small resulting in a high bandgap, and, therefore, deep UV light can be absorbed. The weak bonding of large Tl atom with N atoms results in a low bandgap and weak bonding, and hence the material is IR sensitive.

Since the lattice of N atoms is essentially the same for all of these materials, these Group III element atoms can grade producing a superlattice. This structure can absorb all wavelengths of light within the sensitivity range, with the consequence of moving electrons from the valence band to conduction band. Accordingly, in one aspect of the present invention, the semiconductor layer can be photoreactive, i.e. electron to photon conversion or photon to electron conversion. Thus in one aspect, the semiconductor can be photosensitive and thus react to light and produce a resulting electric current. Many light sensitive devices (e.g. CCDs) are digital, with pixels that can be turned on or off in response to high or low voltage. In one aspect of the present invention, however, an analogue detector can be produced. For example, in a graded semiconductor layer such as (B, Al, Ga, In)N, photons having different energies (i.e. different frequencies or wavelengths) can kick electrons from different layers of the semiconductor with an energy that is related to the acceleration of the electrons, namely voltage. Thus, the pattern of visual excitation received by the semiconductor can generate a pattern of electrons with different voltages at different locations, depending of the angle of the detector relative to the image. Such an analogue detector is essentially a photoreceptor similar in function to the retina of the eye. Integration of the semiconductor with retinal or other neurons through the neuronal growth surface thus creates a system that can generate similar photoreceptive functionality as the retina, and in some cases can be integrated therewith.

Such a device is in contrast to current attempts to implant silicon chips with arrays of electrodes into the eye to allow visual reception in a subject. Such arrays can at best provide a pixilated digital impression of the incoming visual image. However, an analogue chip design, such as that described above, can allow light absorbed on one side to exit as flow of electrons that are different in direction, speed, and flux. Such a pattern may provide the perception of different patterns and shades.

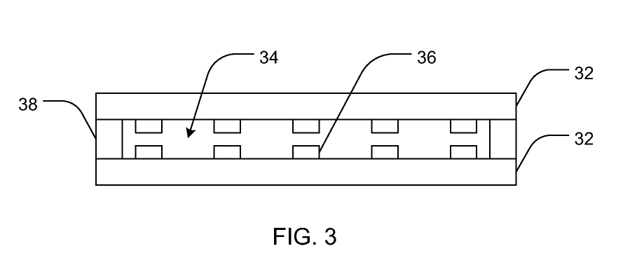

In another aspect of the present invention, a neuronal growth and/or training device is provided. As is shown in FIG. 3, such a device can include a pair of opposing electrode layers 32 spaced apart to create a neuronal growth space 34 there between. Each opposing electrode layer 32 has an electrode array 36 oriented toward the neuronal growth space 34 such that current applied to the electrode array 36 flows across the neuronal growth space 34. Spacer elements 38 can be disposed between the opposing electrode layers 32 to maintain the neuronal growth space 34. Additionally, the device can further include a seal member (not shown) enclosing at least a portion of the neuronal growth space and operable to maintain a liquid in the neuronal growth space.

Neurons grown in the neuronal growth space can connect axons and dendrites across the neuronal space between the array electrodes of the electrode layers in response to stimulation from the arrays. Neurons make connections based, at least in part, on stimulation activity. Those neuronal connections that receive stimulation are maintained, while those that do not receive stimulation are degraded. Thus, in one aspect, the device is a sandwich structure having two planar arrays of electrodes, each of which are registered in a Cartesian coordinate system similar to a front panel display. Patterns of electrical stimulation by the electrode arrays can thus control neuronal connections within the device, forming neuronal networks that are related to the stimulation or training stimulation pattern.

Various configurations of the neuronal training device are possible. For example, in one aspect, two ITO-coated glass sheets can be etched to form parallel lines of conductive circuits. The two sheets are stacked together with glass beads as spacers such that the conductive circuits correspond to one another. A perpendicular circuit matrix is thus formed between the ITO sheets. The gap between the two sheets can then be filled with a growth mixture and a culture of neurons. A stimulating current across the gap between the circuit arrays can thus cause neuronal connections to form, resulting in a neuronal circuit established according to the pattern of stimulation.

Additionally, techniques for growing carbon nanotubes (CNT) with controlled size and location have been developed, and such CNTs can be useful in aspects of the present invention. Typically, CNTs can be formed by decomposition of methane diluted in hydrogen at elevated temperature. A catalyst (e.g. Ni, Co, or Fe) is used to nucleate the CNT, and the diameter of the CNT is controlled by the size of the catalyst. As such, in one aspect, metal conductive lines (e.g. gold) can be formed on a pair of silicon substrates. Each line can be implanted with (e.g. by inkjet) with a liquid containing FeCl₃. The substrate can then be processed in a hydrogen plasma to form iron droplets, and methane introduced at high temperatures can grow CNTs on the iron droplet catalysts. Thus CNTs are formed along the conductive lines across the silicon substrates. CNTs provide a good signal to noise ratio, and neurons can grow between the substrates in accordance with a stimulation pattern. Additionally, CNTs can be either electrically conducting or semiconducting depending on the chirality of the twist of the molecule. The conductor has lower electrical resistance than silver.

In another aspect, multiple neuronal devices can be stacked together to form 3D arrangements. Thus in some aspects, a neuronal-like network can be formed across multiple neuronal devices, as well as within each device across the neuronal space.

One problem that has arisen in the development of computer devices relates to the constant downsizing of electronic circuitry. Further downsizing the circuitry will require the overhaul of the materials designs. For example, the dielectric constant for shielding gates needs to go up, but the dielectric constant for insulating the copper wires needs to go down. Both pose challenges in design and in processing. Furthermore, copper wire size becomes so small that grain boundaries can cause significant variabilities in minute currents passing there through. Consequently, IC gates that are 32 nm or smaller are not only costly, but may face performance limits. Moreover, IC designs are becoming increasingly complex, and heat dissipation has slowed down the clock speed. Many manufacturers are delaying the progression of Moore's Law by spreading computing functions from a single chip to multiple cores, and as such circuitry foot print is used to trade off peak performance.

Such design complexities and the manufacturing difficulties may be solved by using neurons to replace clusters of transistors. In this case, no lithography, depositions, CMP and other laborious procedures are needed. Neuronal networks can be assembled using stimulation patterns according to desired functions. Thus a neuronal network can be created to provide the function of various algorithms, and thus be able to perform computations without the problems associated with thermal management, manufacturing, etc.

### EXAMPLES

The following examples illustrate various techniques of making devices according to aspects of the present invention. However, it is to be understood that the following are only exemplary or illustrative of the application of the principles of the present invention. Numerous modifications and alternative compositions, methods, and systems can be devised by those skilled in the art without departing from the spirit and scope of the present invention. The appended claims are intended to cover such modifications and arrangements. Thus, while the present invention has been described above with particularity, the following Examples provide further detail in connection with several specific embodiments of the invention.

### Example 1

A silicon wafer is seeded with nanodiamond in an ultrasonic bath. The nanodiamond seeded wafer is grown in a microwave plasma CVD reactor that is purged with methane/trimethyl boride, and an abundance of hydrogen. As a result, a layer of boron doped diamond (BDD) is grown on nanodiamond seeds to a thickness of about 10 microns. The BDD is electrically conducting but with a semiconductor bandgap. BDD is subsequently graded to BN/AN/GaN/InN by Atomic Layer Deposition (ALD). This graded material can absorb across the entire spectrum of visible light and some UV. The Si wafer is etched away with concentrated KOH solution to expose the BDD with the surface protruded with high density nuclei (about one trillion per square centimeter) of nanodiamond.

### Example 2

In this example, nitride semiconductors are not used. BDD is back converted to make graphene on the surface. A (100) face can form graphene perpendicular to the surface. Such perpendicular oriented graphene can act as an emitting antenna because graphene has minimal electrical resistance. Graphene can transmit terahertz signals due to its high stiffness. On the other hand, a (111) face can form graphene layers in parallel to the surface by straightening the puckered hexagons of carbon atoms. Such a parallel orientation can be used as a receiver for signals. Graphene is also a piezoelectric layer on diamond surface so radiation signals can be converted to phonons (lattice vibrations) vice versa.

### Example 3

A small single crystal diamond made (twins with triangular shape, typically flat) is etched in oxygen plasma to form a frosted surface with nano-sized protrusions. A boron-doped diamond layer is CVD deposited on the other side of the made from the frosted surface using BCl₃ and CH₄ diluted in H₂. The CH₄ is replaced with NH₃ to grow a layer of cubic BN (cBN). The cBN is doped with C or Si by ion implantation followed by annealing. The result is a p-type boron-doped diamond and n-type cBN structure with nanodiamond protrusion on one side that can be used to anchor neurons.

### Example 4

A Si wafer is immersed in an ethanol bath with nanodiamond particles suspended therein. The bath is ultrasonically agitated to mechanically attach the nanodiamond particles to the Si surface with a high density. A GaAs wafer with a P-N junction is pressed against the Si wafer under vacuum with heating to wafer bond the two wafers together. After the wafer bonding step, the Si wafer is etched away via a NaOH solution. The resulting GaAs wafer has dense (e.g. one billion per square centimeter) nanodiamond protrusions. Neurons can be grown on the nanodiamond surface.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A neuronal growth device, comprising:
a layer of nanodiamond particles having an exposed neuronal growth surface;
a doped diamond layer contacting the layer of nanodiamond particles opposite the neuronal growth surface; and
a semiconductor layer coupled to the doped diamond layer opposite the layer of nanodiamond particles.

2. The device of claim 1, wherein the nanodiamond particles are substantially immobilized by the doped diamond layer.

3. The device of claim 1, wherein the doped diamond layer is a member selected from the group consisting of boron-doped diamond, nitrogen-doped diamond, and combinations thereof.

4. The device of claim 1, wherein the doped diamond layer is a boron-doped diamond layer.

5. The device of claim 1, wherein the semiconductor layer is coupled to the diamond layer with a member selected from the group consisting of braze, solder, and combinations thereof.

6. The device of claim 1, wherein the semiconductor layer is formed on the diamond layer.

7. The device of claim 1, wherein the semiconductor layer is a member selected from the group consisting of GaN, AlN, (B, Al)N, (Ga, In)N, Al₂O₃, and combinations thereof.

8. The device of claim 1, wherein the semiconductor layer is a member selected from the group consisting of GaN, (Ga, In)N, and combinations thereof.

9. The device of claim 1, further comprising a metal layer disposed between the diamond layer and the semiconductor layer.

10. A method of making a neuronal growth substrate, comprising:
forming a layer of nanodiamond particles on a support substrate;
forming a layer of diamond on the layer of nanodiamond particles opposite to the support substrate;
doping the layer of diamond;
coupling a semiconductor layer to the doped diamond layer opposite to the layer of nanodiamond particles;
removing the support substrate from the layer of nanodiamond particles to expose a neuronal growth surface.

11. The method of claim 10, wherein forming the layer of nanodiamond particles on the support substrate further includes:
contacting the support substrate to a solution containing suspended nanodiamond particles; and
ultrasonically agitating the solution to embed the nanodiamond particles on the support substrate.

12. The method of claim 10, wherein forming the layer of nanodiamond particles on the support substrate further includes vapor depositing the layer of nanodiamond particles on the support substrate.

13. The method of claim 10, wherein forming the layer of nanodiamond particles on the support substrate further includes sintering polycrystalline diamond into a polycrystalline diamond layer on the support substrate.

14. The method of claim 10, wherein forming the layer of diamond on the layer of nanodiamond particles includes vapor depositing the diamond layer on the layer of nanodiamond particles.

15. The method of claim 14, wherein doping the diamond layer includes doping the diamond layer with a dopant during vapor deposition of the diamond layer.

16. The method of claim 10, wherein coupling the semiconductor layer to the doped diamond layer includes a member selected from the group consisting of brazing, soldering, and combinations thereof.

17. The method of claim 10, wherein coupling the semiconductor layer to the doped diamond layer includes forming the semiconductor layer on the doped diamond layer.

18. The method of claim 10, further comprising disposing a metal layer on the diamond layer prior to coupling the semiconductor layer.

19. A neuronal training device, comprising:
a pair of opposing electrode layers spaced apart to create a neuronal growth space there between, each opposing electrode layer having an electrode array oriented toward the neuronal growth space such that current applied to the electrode array flows across the neuronal growth space; and
spacer elements disposed between the opposing electrode layers to maintain the neuronal growth space.

20. A neuronal growth device, comprising:
a polycrystalline diamond layer having an exposed neuronal growth surface; and
a semiconductor layer coupled to the polycrystalline diamond layer opposite the neuronal growth surface.
